(19) United States
(12) Patent Application Publication
(10) Pub. No.: US 2024/0358943 A1
(43) Pub. Date: Oct. 31, 2024

(54) VENTILATOR

(71) Applicant: Loewenstein Medical Technology S.A., Luxembourg (LU)

(72) Inventor: Angela GERLACH, Hamburg (DE)

(21) Appl. No.: 18/643,017

(22) Filed: Apr. 23, 2024

(30) Foreign Application Priority Data
Apr. 25, 2023 (DE) 102023110582.5

(51) Int. Cl. A61M 16/00

(52) U.S. Cl. CPC A61M 16/0066; A61M 2205/10; A61M 2205/42

(57) ABSTRACT

A ventilator comprising an inlet, an outlet and a blower, wherein the ventilator forms, between the inlet and the outlet, a respiratory gas path which is routed through the blower. The ventilator, for the routing of the respiratory gas path, further comprises a chamber, a first channel leading into the chamber, and a second channel leading into the chamber. A first acoustic impedance of the first channel and a second acoustic impedance of the second channel deviate from a third acoustic impedance of the chamber such that a sound generated by the blower is attenuated.

### CROSS-REFERENCE TO RELATED APPLICATIONS

The present application claims priority under 35 U.S.C. § 119 of German Patent Application No. 102023110582.5, filed Apr. 25, 2023, the entire disclosure of which is expressly incorporated by reference herein.

### BACKGROUND OF THE INVENTION

### 1. Field of the Invention

The invention relates to a ventilator.

### 2. Discussion of Background Information

A ventilator is a machine for the ventilation of persons with inadequate or suspended spontaneous breathing. Ventilators typically comprise an inlet, an outlet and a blower. The blower serves to convey a respiratory gas. In the context of the present disclosure, a respiratory gas is to be understood as meaning a gas that is conveyed by a ventilator for ventilation purposes. Between the inlet and the outlet, the ventilator forms a respiratory gas path in which the blower can be arranged. The respiratory gas path can be formed, for example, by ducts, channels, chambers and the like, but also routed through functional components of the ventilator, such as the blower.

In ventilators, sound suppression is a major challenge. The pressure necessary for ventilation requires blowers that operate at high speed. These blowers can operate regularly at speeds of up to 60,000 rpm or even higher. However, at correspondingly high speeds, blowers also emit a large amount of sound radiation. This results in a relatively high noise level, which one seeks to reduce. In the prior art, foam is regularly used in combination with a sound insulation structure in order to reduce the operating noise.

For example, EP 1457222 B1, the entire disclosure of which is incorporated by reference herein, discloses a method for sound insulation during ventilation, in which at least part of a respiratory gas is passed through a damping box. The damping box has a flow channel in the region of which the respiratory air is guided past a removable sound-absorbing material. The sound-absorbing material is a foam or a fleece. On account of the foam used, there is an increased outlay in terms of material, and additional components are used in the ventilator.

The material outlay and thus the cost of the ventilator are increased. The use of a sound-absorbing material, as proposed in the prior art, also increases the size of the ventilator, which is considered to be detrimental both for transport reasons and also for reasons of space. In addition, the production of the ventilator also proves more complex, especially since extra installation work is needed for the additional foam components.

In view of the foregoing, it would be advantageous to have available a ventilator which is less complex as regards the use of different materials or components, while at the same time satisfactorily reducing the operating noise of the ventilator. It also would be advantageous to have available a ventilator which provides sound suppression and which is compact and easy to manufacture.

### SUMMARY OF THE INVENTION

The invention provides a ventilator as set forth in the claims.

In particular, a ventilator is provided with an inlet, with an outlet and with a blower, wherein, between the inlet and the outlet, a respiratory gas path is formed which is routed through the blower. A chamber, a first channel leading into the chamber and a second channel leading into the chamber are arranged along the respiratory gas path. Further, a first acoustic impedance of the first channel and a second acoustic impedance of the second channel deviate from a third acoustic impedance of the chamber, such that a sound generated by the blower is attenuated.

The first, second and third acoustic impedances can each be understood as an acoustic flux impedance. The acoustic flux impedance is the resistance that is offered to sound propagation, for example in pipes. A transition between regions with different acoustic impedance gives rise to reflections, which results in a sound-absorbing effect. As has been observed, the design of the ventilator according to the present disclosure, with the chamber and with the first channel and the second channel opening into the chamber, leads to a reduction in the operating noises from the ventilator. This is due to the fact that the third acoustic impedance of the chamber differs from the first acoustic impedance of the first channel and from the second acoustic impedance of the second channel. The first acoustic impedance of the first channel may, but does not necessarily have to, match the second acoustic impedance of the second channel. In other words, there is an impedance mismatch between the first channel and the chamber, and between the second channel and the chamber.

In order to achieve a difference in the acoustic impedance between the chamber and the first or second channel, the chamber and the first or second channel can be dimensioned differently. According to one embodiment of the present invention, the chamber and the first or second channel have a different cross section and/or a different volume. It is advantageous if the respiratory gas path widens at a transition from the first channel or the second channel into the chamber. In particular, a cross section of the respiratory gas path in the first channel or in the second channel can be smaller than within the chamber. According to another embodiment of the present invention, however, it is provided that the cross section of the respiratory gas path in the first channel or in the second channel is larger than within the chamber.

In particular, the respiratory gas path, preferably the first channel and/or the second channel and/or the chamber, more preferably the components of the ventilator along the respiratory gas path, can be formed free of foam and/or free of fleece.

The ventilator preferably has a housing. The inlet and/or the outlet are preferably formed by the housing. Preferably, the blower is arranged within the housing. The respiratory gas path preferably runs within the housing. For this purpose, several channels and/or chambers can be arranged within the housing, for example. According to the present disclosure, the housing, the exterior and interior walls of the ventilator and other components of the ventilator may be formed by various materials, for example by metal and/or plastic.

The blower of the ventilator is a device that can convey a gaseous medium. It can in principle be a ventilating fan, a cooling fan or another type of blower. According to a preferred variant of the present invention, the blower is a radial blower, in particular a radial blower with a semi-open impeller. The respiratory gas path passes through the blower. According to the present invention, a respiratory gas can be received in the region of the inlet and ejected from the outlet.

Within the meaning of the present disclosure, the first channel or the second channel should in principle be understood as a passage that can have any desired shape and/or length. According to the present disclosure, this passage can be formed in any manner by components of the ventilator, for example by internal walls, parts of the housing, tubing, etc. The first channel preferably has an opening into the chamber, which is to be referred to below as a first opening. The second channel preferably has an opening into the chamber, which is to be referred to below as a second opening. According to possible embodiments of the present disclosure, the first channel or the second channel can differ from directly adjacent portions of the respiratory gas path, in particular the chamber, and it does so in particular as regards a size of its cross section orthogonal to the flow direction. It is preferred if a cross section and/or a shape of the respiratory gas path at a transition between the first channel or the second channel and an adjacent portion of the respiratory gas path, in particular the chamber, abruptly decreases or increases.

In a particular embodiment, the first channel or also the second channel can have a minimum length or three-dimensional extent into the volume or into the chamber, in particular also almost no elongate extent. For this purpose, the channels can be designed in particular in slit form. In particular, the channels can be designed in slit form in such a way that the entrance into and the exit from those channels are configured like a kind of slit between the space, lying in front or behind, and the chamber. In particular, the respective length of the respective channel can correspond to the thickness of the wall surrounding the respective channel.

Preferably, the blower is arranged in the chamber. This makes it possible to achieve a compact design, in which the volume of the chamber is utilized to accommodate the blower. In this embodiment, improved sound insulation was observed. There are different explanations for this: First, the blower is additionally shielded from the environment of the ventilator on account of the fact that it is located inside the chamber. Furthermore, sound waves emitted by the blower are attenuated by the structure of chamber, first channel and second channel according to the operating principle described above.

A further advantage of this embodiment is that heat can also be dissipated from the blower externally by convection, rather than just internally. The blower is preferably arranged completely in the chamber; supply lines of the blower, such as hoses and the like, are not to be interpreted as being part of the blower. According to possible embodiments of the present disclosure, these lines can also be located outside the chamber or can be arranged in sections in a wall of the chamber, for example. The blower is preferably arranged in the chamber in such a way that a respiratory gas can flow around it on several sides, which can serve for better cooling. According to an advantageous variant of the present invention, the blower is not surface-mounted on a wall of the chamber, and/or the blower or a portion of the blower is not part of a wall or of several walls of the chamber. According to the present invention, the blower can be arranged spaced apart from one or more walls of the chamber. For example, the blower can be held or mounted away from the wall by one or more retaining elements. Preferably, the blower within the chamber does not have an opening for receiving or ejecting the respiratory gas.

The blower body is preferably fastened or suspended in the chamber. It is particularly preferred if the blower body is fastened in the chamber in a vibration-damping manner. Preferably, the blower body is fastened in the chamber via a vibration-damping material. The vibration-damping material can be an elastomer according to the present disclosure. The vibration-damping material is preferably silicone. Preferably, the blower body is fastened in the chamber via three suspension points. In this case, according to one embodiment of the present invention, two of the suspension points of the blower body can be fastened to holders, and, furthermore, the third suspension point of the blower body can be fastened to a pressure nozzle. The vibration-damping material is preferably arranged between the holders and the blower body or between the pressure nozzle and the blower body. The vibration-damping suspension or fastening of the blower causes the blower body to vibrate less. Moreover, vibrations of the blower body are transmitted to the housing of the ventilator only in an attenuated manner. This further reduces noise emissions of the ventilator.

The chamber can have any cross-sectional shape. It can be rectangular according to the present invention. However, it does not need to be angular and, according to one embodiment of the present invention, can be round on the inside. Both long and short configurations of the chamber are conceivable according to the present invention. However, it is advantageous if the chamber has a volume of about 100 cm³-5000 cm³, preferably about 500 cm³-3000 cm³, particularly preferably about 600-2000 cm³ or about 100-2000 cm³. It has proven particularly advantageous if the chamber has a volume of about 500-1200 cm³.

Preferably, the respiratory gas path between the inlet and the chamber is formed at least in part by the first channel. The first channel is thus arranged between the inlet and the chamber. Starting from the inlet, a respiratory gas is thus routed through the first channel into the chamber. Furthermore, the respiratory gas path between the outlet and the chamber can be formed at least in part by the second channel. The second channel is thus arranged between the outlet and the chamber. The respiratory gas can thus flow from the chamber to the outlet through the second channel. Furthermore, according to the present disclosure, it can be provided that the respiratory gas path between the second channel and the outlet is routed through the blower. Thus, a respiratory gas emerging from the chamber passes first through the blower before reaching the outlet. The blower can thus convey the respiratory gas along the respiratory gas path. Alternatively, however, it can also be provided that the blower is arranged along the respiratory gas path between the inlet and the first channel.

According to a particular embodiment of the present invention, the second channel opens into an auxiliary chamber, wherein a fourth acoustic impedance of the auxiliary chamber deviates from the second acoustic impedance of the second channel, such that the sound generated by the blower is additionally attenuated. The auxiliary chamber can have any desired shape. It is advantageous if the auxiliary chamber has a volume of about 10-200 cm³. A volume of about 30-100 cm³ has proven particularly advantageous. An opening of the blower is preferably connected to the auxiliary chamber directly or via a third channel. The opening of the blower is preferably an inlet of the blower. For example, the third channel can be a hose or a pressure nozzle. According to a variant of the present disclosure, the hose or the pressure nozzle can be formed in part by a sound-absorbing material or can be connected to the blower via a sound-absorbing material.

The chamber and the auxiliary chamber preferably adjoin each other. A compact design can be achieved in this way. According to a variant of the present disclosure, the chamber is arranged on one side of a wall of the ventilator, and the auxiliary chamber is arranged on another side of this wall. According to the present disclosure, it can be provided that both the first channel and the second channel are routed through this same wall. In addition, the third channel can be routed through this wall. It can also be provided that the blower body is fastened to this same wall. The wall is preferably a base plate of the chamber.

According to an alternative embodiment of the present invention, the second channel is attached directly to the opening of the blower. There is therefore no auxiliary chamber or other cavity between an output of the second channel and the opening of the blower. According to the present disclosure, it is possible that the second channel is routed out of the chamber and is attached to an opening of the blower arranged outside the chamber. Alternatively, it is possible that the second channel is arranged completely inside the chamber and is attached to an opening of the blower arranged inside the chamber. The second channel is therefore not routed out of the chamber through a wall.

According to one embodiment, the first channel and/or the second channel can extend into the chamber. This means that the first channel and/or the second channel protrudes into the chamber. According to the present invention, the first channel and the second channel can be routed through a wall that delimits the chamber. According to an alternative embodiment of the present invention, the first channel and/or the second channel can already terminate in the wall, such that it does not protrude into the chamber at all.

According to one embodiment, the first channel and the second channel can extend protruding into the chamber, wherein the first channel and the second channel preferably extend protruding into the chamber to different extents, are oriented differently and/or extend protruding into the chamber from different side walls. According to an advantageous embodiment of the present invention, the first channel and the second channel are oriented parallel to each other. Alternatively, the first channel and the second channel can be at an angle of about 90° to each other. However, the first channel and the second channel can also enclose any other angle. Thus, different arrangements of the first channel and of the second channel are conceivable. It is also possible according to the present invention that the first channel and the second channel have different shapes.

A position, a shape and a size of the first channel and of the second channel can be selected depending on a size of the chamber, a shape of the chamber and/or a position of the blower. This is particularly advantageous in embodiments in which the blower is arranged within the chamber. An appropriate arrangement can also be used to achieve improved flow control within the chamber, such that flow noise is reduced. In particular, the aim should be to ensure that a flow velocity of the respiratory gas between the first channel and the second channel does not become too high. To prevent this, the openings of the first channel and of the second channel should preferably not be directly aligned with each other, and/or the channels should extend to different extents into the chamber. This is especially true if the first channel and the second channel are arranged relatively close to each other.

According to the present invention, it is moreover possible that the first channel and the second channel extend into the chamber, wherein the first opening of the first channel and the second opening of the second channel are arranged such that, during operation of the ventilator, a respiratory gas flow is established that flows at least in part around the blower arranged inside the chamber. Thus, the blower is cooled by the respiratory gas flowing around it. To achieve this, the first channel and the second channel can be dimensioned and/or oriented accordingly. According to this variant of the present invention, the blower body is preferably located between the first opening of the first channel and the second opening of the second channel.

The first channel and/or the second channel preferably extends outside the chamber from the wall of the chamber. The first channel or the second channel is thus routed out of the chamber on the outside. In this embodiment, the first channel and/or the second channel can nevertheless also extend into the chamber. Thus, compact designs are possible, for example in the case where the chamber is to be comparatively flat. Then a portion of the first channel and/or of the second channel can be arranged outside the chamber. According to the present invention, the first channel and the second channel outside the chamber can have different lengths, different dimensions and/or different orientations. According to advantageous embodiments of the present invention, the first channel outside the chamber forms the inlet of the ventilator. According to the present disclosure, it can also be provided that the second channel outside the chamber forms the outlet of the ventilator.

According to one embodiment, the first channel and/or the second channel can have a curved or undulating profile. The first channel or the second channel with a curved profile is preferably curved to the side, for example in a banana shape or arc shape. As a result, the opening of the first channel or of the second channel into the chamber is preferably oriented laterally. It is thus possible to avoid a situation where a respiratory gas emerging from the channel strikes a wall of the chamber perpendicularly. In terms of flow technology, it is advantageous that this is avoided in order to reduce flow noise. With a corresponding orientation of the opening of the first channel or of the second channel, an undulating profile has a corresponding advantage, further that the first channel or the second channel can be routed around obstacles within the chamber.

According to one embodiment, the first channel and/or the second channel can have a cross-sectional area orthogonal to a longitudinal direction of the first channel or of the second channel, which cross-sectional area is curved. According to the present disclosure, the cross-sectional area can be crescent-shaped or have another kind of curved profile. Advantages in terms of flow dynamics can be achieved in this way. For example, the respiratory gas flow can be routed around any components arranged in the chamber, in order to reduce flow noise.

According to one embodiment, the first channel and/or the second channel can form a volume with a rectangular cross section, in particular a substantially square volume. However, different shapes are also possible. The volume preferably has a depth of about 1-50 mm, preferably about 1-20 mm, particularly preferably about 3-7 mm, a width of about 5-100 mm, preferably about 20-60 mm, and a length of about 1-100 mm, preferably about 20-60 mm.

This results in much reduced sound emission from the ventilator. This applies in particular when the chamber or the auxiliary chamber is dimensioned as defined above. The length of the volume is understood as an extent of the volume in the direction of flow. The width and depth of the volume span a cross-sectional area of the volume that is perpendicular to the direction of flow.

According to a further embodiment of the present invention, the first channel and/or the second channel are designed to narrow or widen in the direction of an opening of the channel into the chamber. Preferably, the opening angle is not greater than about 20°. Thus, a profile of the respiratory gas flow can be adjusted in order to achieve a reduction of the flow noise. Furthermore, this embodiment of the present disclosure has manufacturing advantages.

It is advantageous if the first channel and/or the second channel have/has rounded inner edges. This has manufacturing advantages. Alternatively, however, it is also possible that the first channel and/or the second channel have/has angular inner edges, in particular inner edges in which wall pieces are oriented at an angle of about 90° to each other. According to the present disclosure, it can also be provided that ribs, in particular longitudinal ribs, are arranged in the first channel and/or in the second channel, by which it is possible to achieve advantages in term of flow dynamics and manufacturing.

In a simplified representation of the actual geometries, the channels or slits have cross sections and circumferences that can be determined by their depth and their width. For example, the cross section of the channels can be rectangular or oval with a high height-width ratio. Other shapes are also conceivable, and the cross sections can have an overall curved or angular shape or another irregular shape. The first channel and the second channel can have identical or different cross-sectional shapes. The cross-sectional shape is oriented perpendicular to the direction of flow. The cross-sectional shape of the channels can be selected at least in part from the following shapes: rectangular, slit-shaped, crescent-shaped, oval, cross-shaped, X-shaped, star-shaped, round or angular annular gap, L-shaped, dumbbell-shaped, U-shaped, V-shaped, T-shaped, ellipsoid, trapezoid, round. The cross-sectional shapes of the first channel and of the second channel can be identical or different. In preferred embodiments, the channels can have a substantially smooth inner surface.

The cross-sectional shapes of the channels should be chosen in such a way that they each meet their acoustic requirements. To this end, it is advantageous if the channels have the least possible depth and yet can hold a certain volume. Regarding the second channel, it must be remembered that it is arranged adjacent to the suction nozzle of the blower or opens into the intake chamber of the blower—the auxiliary chamber. The flow arriving at the blower should advantageously be as symmetrical as possible. For this reason, it is advantageous if the geometry of the second channel transitions as symmetrically as possible into the auxiliary chamber. The cross-sectional shape of the second channel is thus preferably rectangular or slit-shaped with a small depth.

In relation to the first channel, it is more important that there is a free length in the direction of flow behind the first channel, such that the flow from the first channel flows freely into the chamber and does not directly strike a wall. The geometry of the first channel is less critical with respect to the flow and can be selected more variably. The cross-sectional shape of the first channel, like the second channel, can be slit-shaped. In preferred embodiments, however, the cross-sectional shape of the first channel can also be selected in such a way that a more compact design can be realized. This can be achieved by geometries with a smaller width. It should be stressed that the first channel and the second channel are not part of a pressure differential sensor.

For example, at least two such channels are arranged in the respiratory gas path, and these channels can be connected in series and by an intermediate chamber or auxiliary chamber. The two channels can basically have the same cross section, or the cross sections can be different.

The relative size of the cross-sectional areas and of the corresponding perimeters can be characterized by the
ratio R of the
square perimeter P to the
area A:

$$R = {P}^{2} / A$$

This ratio R has, for at least one channel or slit, preferably a value of over about 14, preferably about 16 or more, particularly preferably about 20 or more, particularly preferably about 25 or more, more preferably about 30 or more, for example also 30, 35, 40, 45 or 50. Laboratory tests for different R-values are given in the following table:

| Channel type | Sound attenuation | R = P²/A |
|---|---|---|
| K1 | −− | 12-14 |
| K2 | + | 16-20 |
| K3 | + | 21-25 |
| K4 | + | >25 |
| K5 | − | >40 |

In a preferred embodiment, the transition between one or both channels and the chamber is abrupt, which means that there is significant geometric discontinuity.

In some embodiments, the channels have wider end portions, for example. This offers the advantage of reducing the flow loss.

A filter chamber for accommodating a removable filter component can be positioned in the respiratory gas path directly upstream of a channel.

One or more channels can be substantially straight or curved in the direction of flow.

One or both channels can be divided into several parallel sub-channels. The cross-sectional circumference of each channel is then evaluated as the sum of the cross-sectional circumference of its sub-channels. Similarly, the cross-sectional area is evaluated as the sum of the cross-sectional areas of its sub-channels.

In one embodiment, at least one channel has a cross-sectional area A and a perimeter P which are characterized by the ratio R of the square perimeter P to the area A according to the formula R=P²/A

wherein this ratio R assumes a value of over about 14, preferably about 16 or over about 16, more preferably about 20 or over about 20, still more preferably about 30 or over about 30.

### BRIEF DESCRIPTION OF THE DRAWINGS

The invention will be described in more detail by way of example on the basis of FIGS. 1 to 11, in which:

FIG. 1 shows an exterior view of a first embodiment of the ventilator according to the present invention,

FIG. 2 shows a sectional view of the first embodiment of the ventilator according to the present invention,

FIG. 3 shows a partial view of the first embodiment of the ventilator according to the present invention,

FIG. 4 shows a partial view of a second embodiment of the ventilator according to the present invention, where a first channel of the ventilator has a curved profile,

FIG. 5 shows a partial view of a third embodiment of the ventilator according to the present invention, where a first channel and a second channel of the ventilator have cross-sectional areas that are curved,

FIG. 6 shows a partial view of a fourth embodiment of the ventilator according to the present invention, where a first channel and a second channel of the ventilator are designed such that they widen in the direction of their openings into a chamber of the ventilator,

FIG. 7 shows a partial view of a fifth embodiment of the ventilator according to the present invention, where a first channel of the ventilator is designed such that it widens in the direction of its opening into a chamber of the ventilator, and the first channel is additionally curved,

FIG. 8 shows a partial view of a sixth embodiment of the ventilator according to the present invention, where a first channel and a second channel of the ventilator have longitudinal ribs, and

FIG. 9 shows a partial view of a seventh embodiment of the ventilator according to the present invention, where a first channel and a second channel of the ventilator extend outside a chamber of the ventilator from a wall of the chamber.

FIG. 10 and FIG. 11 show schematic views of different embodiments of the cross-sectional shapes of the first channel and second channel.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

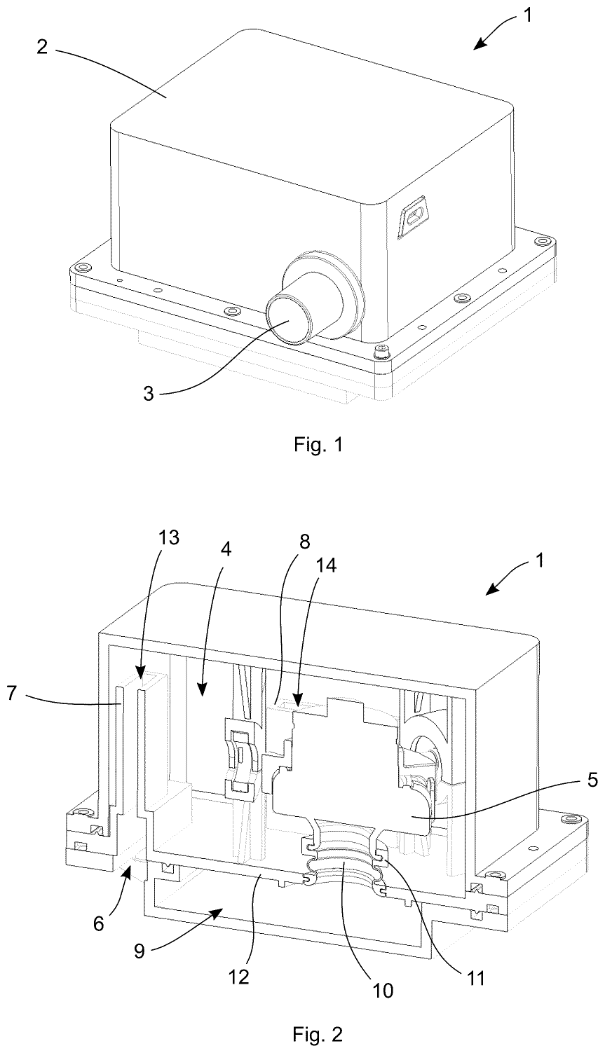

FIG. 1 shows an exterior view of a first embodiment of the ventilator 1 according to the present invention. The ventilator 1 has a housing 2. The ventilator 1 ejects a respiratory gas from an outlet 3. The ventilator 1 moreover has an inlet, which is arranged on an underside of the ventilator 1 and is not visible in FIG. 1.

FIG. 2 shows a sectional view of the first embodiment of the ventilator 1 according to the present invention. The ventilator 1 forms a chamber 4. A blower 5 of the ventilator 1 is arranged in the chamber 4. The blower 5 is a radial blower.

The ventilator 1 has an inlet 6 through which the respiratory gas can enter the ventilator 1. The respiratory gas entering the ventilator 1 through the inlet 6 follows the following respiratory gas path within the ventilator 1: First, the respiratory gas flows through a first channel 7 of the ventilator 1. The respiratory gas then enters the chamber 4. The respiratory gas flows through the chamber 4 and subsequently enters a second channel 8. The respiratory gas passes through the second channel 8 into an auxiliary chamber 9. From the auxiliary chamber 9, the respiratory gas is sucked through a nozzle 10 of the ventilator 1 by the blower 5. The blower 5 conveys the respiratory gas onward, such that it finally exits from the outlet (not shown in FIG. 2) of the ventilator 1.

An end portion 11 of the nozzle 10 is made of silicone. Thus, the blower 5 is connected to a base plate 12 of the chamber 4 in a vibration-damping manner.

A first acoustic impedance of the first channel 7 and a second acoustic impedance of the second channel 8 deviate from a third acoustic impedance of the chamber 4. Therefore, sound reflections occur at a transition from a first opening 13 of the first channel 7 into the chamber 4 and at a transition from a second opening 14 of the second channel 8 into the chamber 4. Thus, a sound generated by the blower 5 is attenuated. This reduces the operating noise of the ventilator 1.

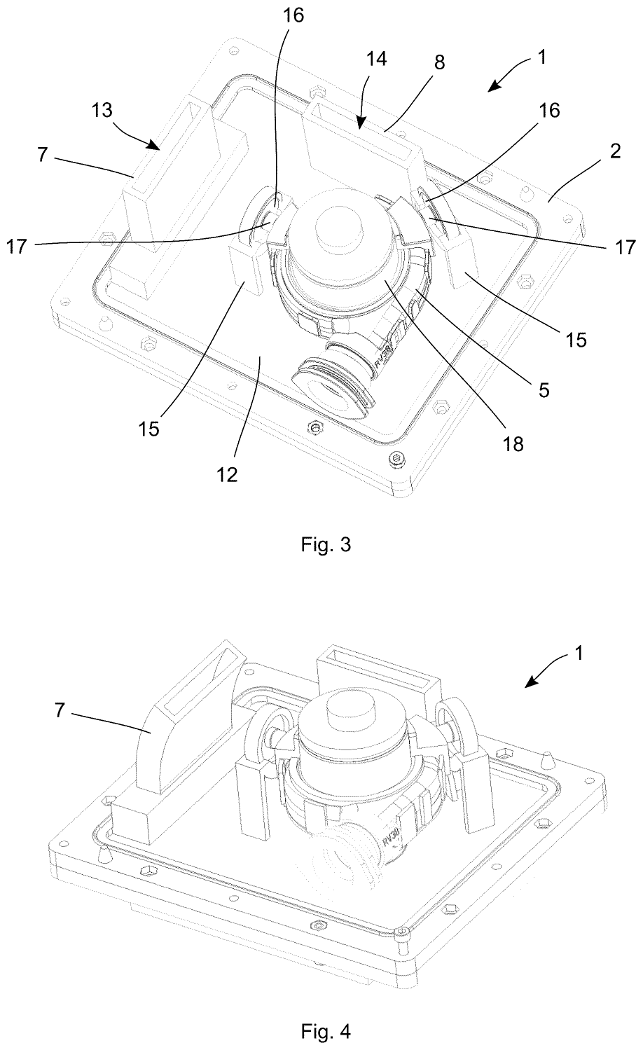

FIG. 3 shows a partial view of the first embodiment of the ventilator 1 according to the present disclosure. In this partial view, and in all subsequent partial views of ventilator 1, an upper housing portion is not shown, and therefore an interior of the chamber is more clearly visible.

The first channel 7 and the second channel 8 are oriented at right angles to each other. In the present case, the first channel 7 and the second channel 8 are arranged such that there are no obstacles located between their openings 13 and 14. This serves to avoid flow noise.

In the present embodiment, two holders 15 are mounted on the base plate 12 and serve for the additional fastening of the blower 5 in the ventilator 1. In end regions, the holders 15 have silicone bearings 16 in which retaining pins 17 of the blower 5 are located. The retaining pins 17 are connected to a blower body 18 of the blower 5. The blower 5 is thus fastened both to the holders 15 and to the nozzle 10 in a vibration-damping manner. This has an additional sound-damping effect and in particular reduces sound transmission by vibrations from the blower body 5 to the housing 2 of the ventilator 1.

FIG. 4 shows a partial view of a second embodiment of the ventilator 1 according to the present disclosure, where a first channel 7 of the ventilator 1 has a curved profile. On account of the curved profile, a respiratory gas flow emerging from the first channel 7 does not strike perpendicularly against an upper wall (not shown here in FIG. 4) of the ventilator 1. This is advantageous in terms of flow dynamics, since flow noise is thus reduced.

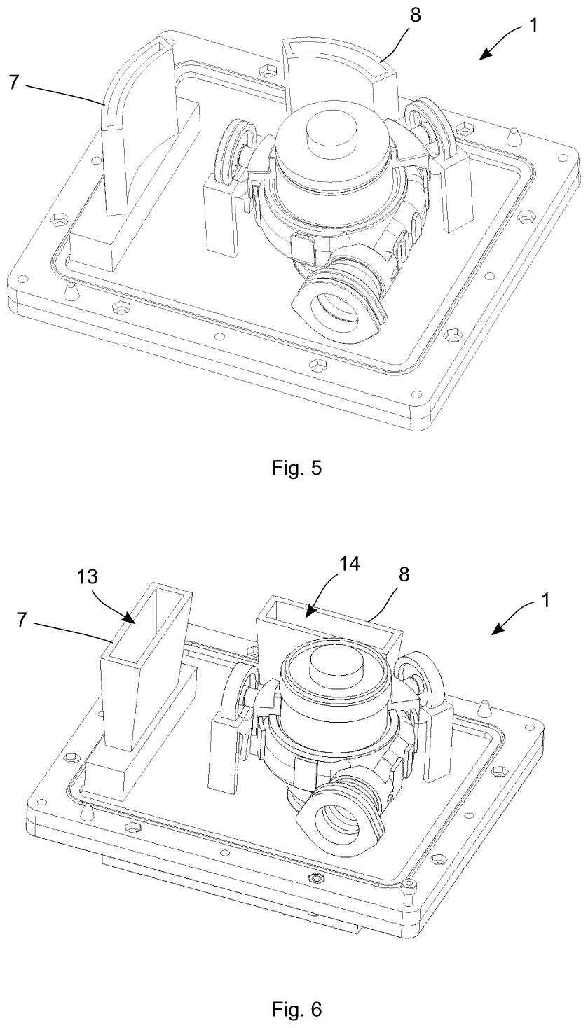

FIG. 5 shows a partial view of a third embodiment of the ventilator 1 according to the present disclosure, where a first channel 7 and a second channel 8 of the ventilator 1 have cross-sectional areas that are curved. The first channel 7 and the second channel 8 protrude into a chamber of the ventilator 1 to different extents. In this way, an advantageous course of the respiratory gas flow in the chamber can be achieved in order to reduce flow noise.

Based on the exemplary embodiment shown in FIG. 5, further exemplary embodiments can also be provided which comprise a different form of curvature of the curved channels. Thus, to mention just one example, an S-shaped cross section, in the sense of an “S stroke”, is also conceivable as a curvature of the channels.

It is likewise possible that one of the two channels has one form of curvature, while the other of the two channels has a different form of curvature.

FIG. 6 shows a partial view of a fourth embodiment of the ventilator 1 according to the present disclosure, where a first channel 7 and a second channel 8 of the ventilator 1 are designed such that they widen in the direction of their openings 13 and 14 into a chamber of the ventilator 1. This serves to advantageously adjust the respiratory gas flow in order to achieve a reduction in the flow noise.

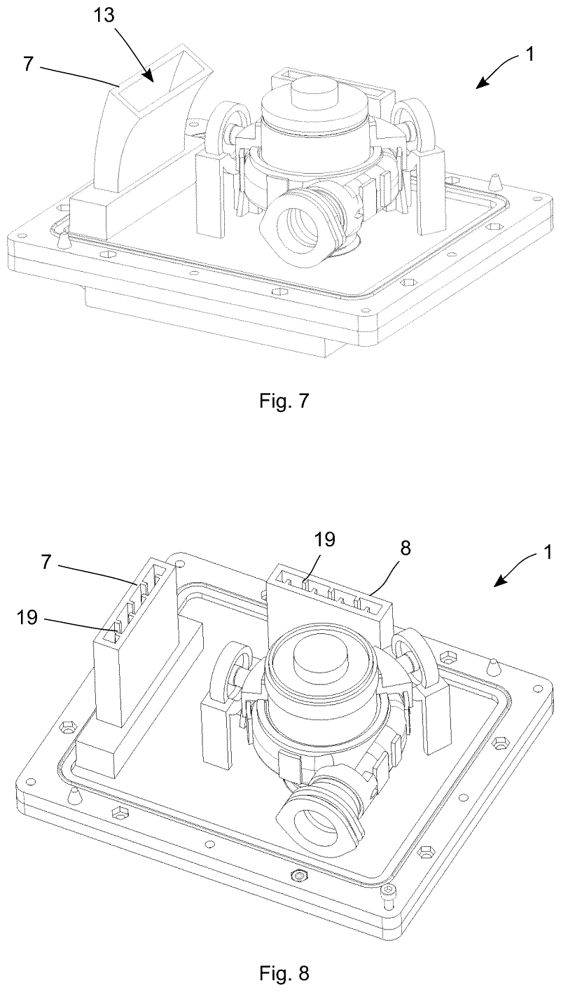

FIG. 7 shows a partial view of a fifth embodiment of the ventilator 1 according to the present disclosure, where a first channel 7 of the ventilator 1 is designed such that it widens into the chamber in the direction of a first opening 13 of the first channel 7, and wherein the first channel 7 is additionally curved. Thus, advantages of the third embodiment and of the fourth embodiment are combined in order to achieve a reduction in the flow noise.

FIG. 8 shows a partial view of a sixth embodiment of the ventilator 1 according to the present disclosure, where a first channel 7 and a second channel 8 of the ventilator 1 have longitudinal ribs 19. This has advantages in terms of flow dynamics and manufacturing technology.

Based on the illustrative embodiment shown in FIG. 8, further illustrative embodiments may also be provided which comprise a different shape of the ribs, which are formed in FIG. 8 as longitudinal ribs 19. Thus, to mention just one example, transverse ribs are also conceivable. It is likewise possible to provide different or combined shapes of ribs, be it transverse ribs or longitudinal ribs or the combination of transverse ribs and longitudinal ribs. To mention one example, the ribs can also be formed in an airfoil shape. Furthermore, it is possible to provide the ribs only in a part of the longitudinal extent of the corresponding channel and therefore not to form the ribs along the full length of the channel.

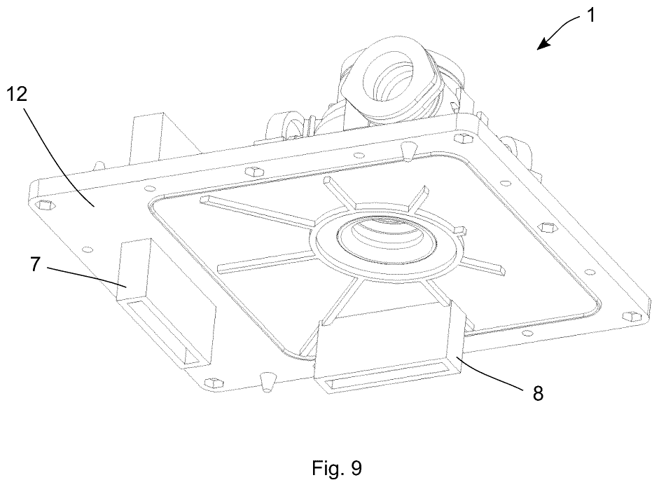

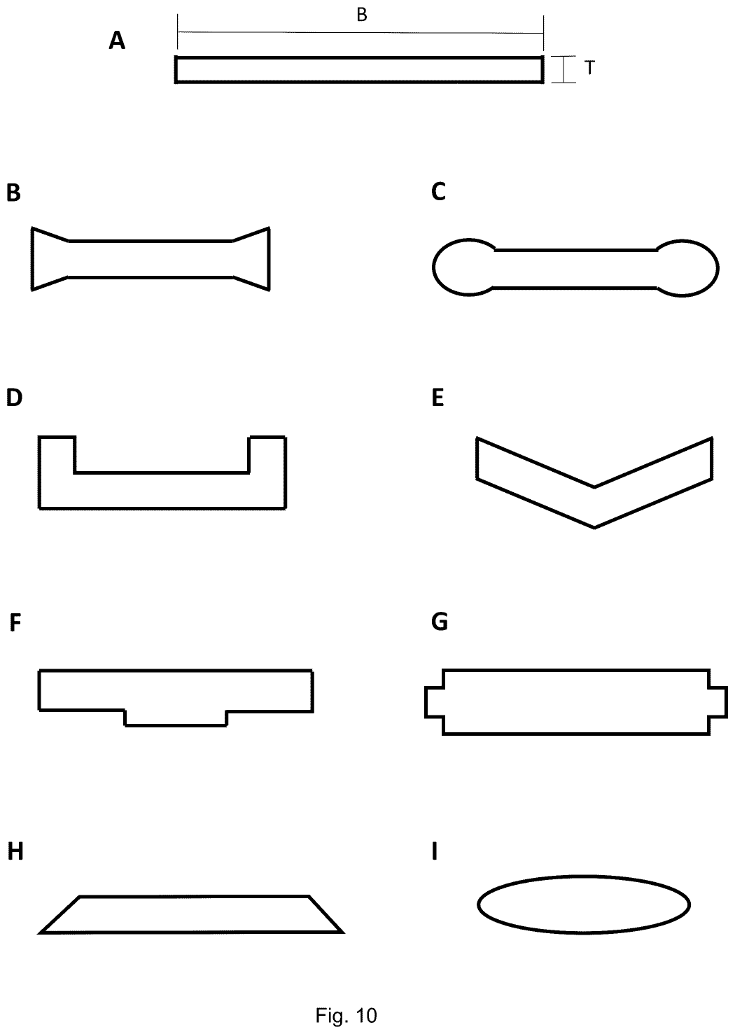

FIG. 9 shows a partial view of a seventh embodiment of the ventilator 1 according to the present disclosure, where a first channel 7 and a second channel 8 of the ventilator 1 extend outside a chamber of the ventilator 1 from a wall of the chamber, wherein the wall is a base plate 12 of the chamber. The first channel 7 and the second channel 8 thus protrude on both sides from the base plate 12. This permits a shallower design of the chamber of the ventilator 1.

Based on the exemplary embodiment shown in FIG. 2 with the auxiliary chamber 9, and on the exemplary embodiment shown in FIG. 9 with channels extending outside a chamber (such as the chamber 4 according to FIG. 2), further exemplary embodiments may also be provided which have a first channel opening into the chamber having the blower (such as the chamber 4 with blower 5 as in FIG. 2), but in which the second channel is arranged only in the auxiliary chamber (for example in the auxiliary chamber arranged below the blower). In such an embodiment, further channels can also be arranged in the auxiliary chamber. Furthermore, it is conceivable that, for example, the first channel is formed only in a slit shape and merely constitutes a kind of slit or aperture into the chamber having the blower.

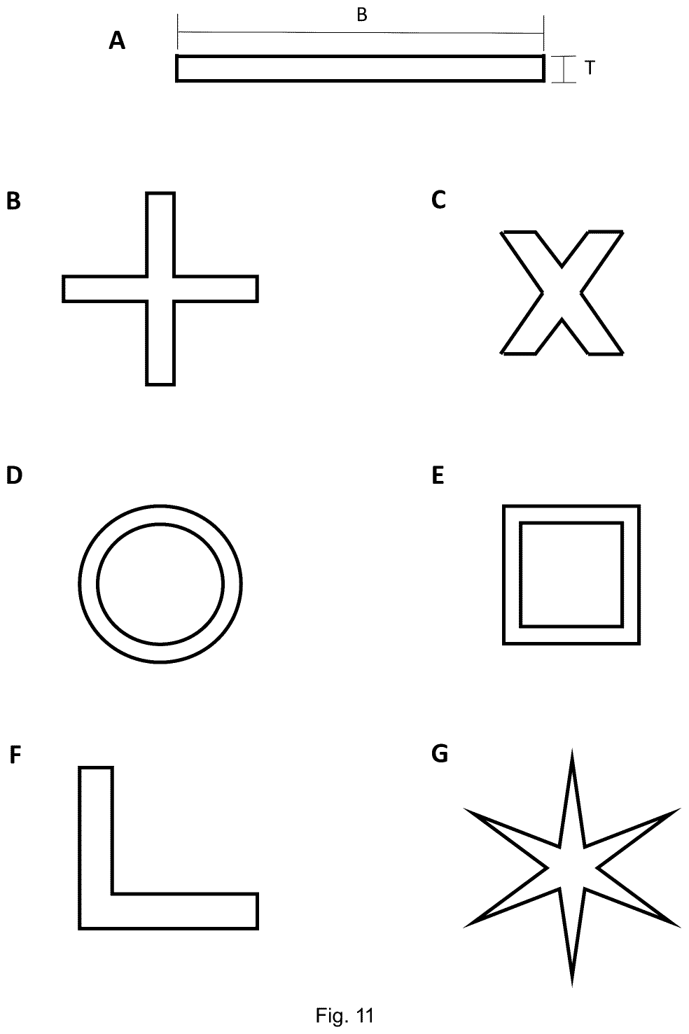

FIG. 10 and FIG. 11 show schematic views of different embodiments of the cross-sectional shapes of the first channel and second channel. The cross-sectional area orthogonal to the longitudinal direction of the channel is shown. The first channel and the second channel can have identical cross-sectional shapes or different cross-sectional shapes.

FIG. 10 shows exemplary embodiments of the cross section suitable for the first channel and second channel. FIG. 10A shows a rectangular cross-sectional shape having a depth T and a width B. The depth T can be many times smaller than the width B, such that the respective channel is slit-shaped. The channel can additionally be curved (not shown here, see FIG. 5), resulting in a crescent-shaped cross section.

In addition to a rectangular cross-sectional shape, other cross-sectional shapes are also conceivable, examples of which are shown in FIGS. 10B-I. FIG. 10B and FIG. 10C show dumbbell-shaped cross-sectional shapes. A dumbbell-shaped cross-sectional shape can be understood as meaning that the channel is slit-shaped in cross section and has a widened region at least at one end, preferably at both ends. The widened regions can be angular (see FIG. 10B) or round (see FIG. 10C). The advantage of dumbbell-shaped cross-sectional shapes is that the widened regions at the ends make the flow profile more uniform than in the case of a slit without widened regions. This can be realized by the larger flow cross section at the respective ends, which can compensate for a speed-reducing friction effect at the ends. FIG. 10D shows a U-shaped cross-sectional shape which, by virtue of the widened regions at the ends, can have similar advantages to those mentioned above. Other possible cross-sectional shapes can be a V-shape (FIG. 10E), a T-shape (FIG. 10F), a double T-shape (FIG. 10G), a trapezoid (FIG. 10H) or an ellipse (FIG. 10I).

FIG. 11 shows exemplary embodiments of the cross section that may be suitable and advantageous in alternative embodiments, in particular for the first channel. In addition to the purely rectangular slit shape (FIG. 11A), other cross-sectional shapes are also conceivable, examples of which are shown in FIGS. 11B-G. FIG. 11B shows a cross-shaped, FIG. 11C an X-shaped, FIG. 11F an L-shaped and FIG. 11G a star-shaped cross-sectional shape. With these cross-sectional shapes, the installation space can be minimized by virtue of an overall smaller width B.

FIGS. 11D and 11E show a round and an angular annular gap, respectively. A triangular or polygonal annular gap is also conceivable (not shown). These cross-sectional shapes are characterized by the flow between the outer circle or square and the inner circle or square. From a technical point of view, such shapes correspond to a “wound-up slit”, which also entails a significant saving in terms of the installation space.

All of the geometries shown in FIGS. 11B-G are characterized in that the corresponding channel has a more compact construction than the slit shape according to FIG. 11A, with a constant cross-sectional area. The total volume, formed by the depth, width and length of the channels, is preferably the same in all cross-sectional shapes.

The length of the channel can be unaffected by the possible cross-sectional shapes according to FIGS. 10 and 11. The length of the channel can be about 1-100 mm, preferably about 20-60 mm, particularly preferably 40 mm. Important in all of the cross-sectional shapes is the depth T. The latter should be the same, at least for the partial shape most similar to the slit, namely 10 about 1-20 mm, preferably about 3-7 mm, particularly preferably about 5 mm. The cross-sectional area, formed by the depth and the width of the channels, is preferably the same in all of the cross-sectional shapes. The cross-sectional area can be about 5-2000 mm², preferably about 60-420 mm², particularly preferably about 200 mm².

### LIST OF REFERENCE SIGNS

1 ventilator
2 housing
3 outlet
4 chamber
5 blower
6 inlet
7 first channel
8 second channel
9 auxiliary chamber
10 nozzle
11 end portion
12 base plate
13 first opening
14 second opening
15 holder
16 silicone bearing
17 retaining pin
18 blower body
19 longitudinal rib

What is claimed is:

1. A ventilator, wherein the ventilator comprises an inlet, an outlet, and a blower, wherein, between the inlet and the outlet, a respiratory gas path is formed which is routed through the blower, wherein a chamber, a first channel leading into the chamber, and a second channel leading into the chamber are arranged along the respiratory gas path, and wherein a first acoustic impedance of the first channel and a second acoustic impedance of the second channel deviate from a third acoustic impedance of the chamber, such that a sound generated by the blower is attenuated.

2. The ventilator of claim 1, wherein the blower is arranged in the chamber, and wherein a blower body of the blower is fastened in the chamber in a vibration-damping manner.

3. The ventilator of claim 1, wherein the chamber has a volume of from 100 cm³ to 5000 cm³.

4. The ventilator of claim 1, wherein the chamber has a volume of from 500 cm³ to 3000 cm³

5. The ventilator of claim 1, wherein the respiratory gas path between the inlet and the chamber is formed at least in part by the first channel.

6. The ventilator of claim 1, wherein the respiratory gas path between the outlet and the chamber is formed at least in part by the second channel.

7. The ventilator of claim 1, wherein the respiratory gas path between the second channel and the outlet is routed through the blower.

8. The ventilator of claim 1, wherein the second channel leads into an auxiliary chamber, wherein a fourth acoustic impedance of the auxiliary chamber deviates from the second acoustic impedance of the second channel such that the sound generated by the blower is additionally attenuated, the auxiliary chamber having a volume of from 10 cm³ to 200 cm³.

9. The ventilator of claim 8, wherein the auxiliary chamber has a volume of from 30 cm³ to 100 cm³.

10. The ventilator of claim 1, wherein the first channel and/or the second channel extends protruding into the chamber.

11. The ventilator of claim 1, wherein the first channel and the second channel extend protruding into the chamber to different extents, are oriented differently and/or extend protruding into the chamber from different side walls.

12. The ventilator of claim 1, wherein the first channel and the second channel extend protruding into the chamber, a first opening of the first channel and a second opening of the second channel being arranged such that, during operation of the ventilator, a respiratory gas flow is established that flows at least in part around the blower.

13. The ventilator of claim 1, wherein the first channel and/or the second channel extends outside the chamber from a wall of the chamber.

14. The ventilator of claim 1, wherein the first channel and/or the second channel has a curved or undulating profile.

15. The ventilator of claim 1, wherein the first channel and/or the second channel has a cross-sectional area orthogonal to a longitudinal direction of the first channel or of the second channel, respectively, the cross-sectional area having, at least in part, a shape selected from rectangular, slit-shaped, crescent-shaped, oval, cross-shaped, X-shaped, star-shaped, a round or angular annular gap, L-shaped, dumbbell-shaped, U-shaped, V-shaped, T-shaped, ellipsoid, trapezoid.

16. The ventilator of claim 1, wherein cross-sectional shapes of the first channel and of the second channel are identical.

17. The ventilator of claim 1, wherein cross-sectional shapes of the first channel and of the second channel are different.

18. The ventilator of claim 1, wherein the first channel and/or the second channel have a volume with a depth of from 1 mm to 50 mm, a width of from 5 mm to 100 mm, and a length of from 1 mm to 100 mm.

19. The ventilator of claim 1, wherein the first channel and/or the second channel have a volume with a depth of from 1 mm to 20 mm, a width of from 20 mm to 60 mm, and a length of from 20 mm to 60 mm.

20. The ventilator of claim 1, wherein at least one channel has a cross-sectional area (A) and a perimeter (P), and ratio (R) of the square perimeter (P) to the cross-sectional area (A) according to the formula R=P²/A
wherein (R) has a value of over 14.
